(19) United States
(12) Patent Application Publication
(10) Pub. No.: US 2009/0076050 A1
(43) Pub. Date: Mar. 19, 2009

(54) SYNERGISTIC TUMOR-KILLING EFFECT OF RADIATION AND BERBERINE COMBINED TREATMENT IN LUNG CANCER

(75) Inventors: Fen-Pi Chou, Taichung (TW); Pei-Ling Peng, Taichung (TW); Hsien-Chun Tseng, Taichung (TW)

Correspondence Address: WPAT, PC;INTELLECTUAL PROPERTY ATTORNEYS, 2030 MAIN STREET, SUITE 1300, IRVINE, CA, 92614, US

(73) Assignee: CHUNG SHAN MEDICAL U., Taichung (TW)

(21) Appl. No.: 12/184,733

(22) Filed: Aug. 1, 2008

Related U.S. Application Data
(60) Provisional application No. 60/954,164, filed on Aug. 6, 2007.

(51) Int. Cl. A61K 31/4375; A61P 35/00

(52) U.S. Cl. 514/280

(57) ABSTRACT

A method for treating a subject suffering cancer, comprising administering an effective amount of berberine or its acid or ester derivates to the subject in need of such treatment, and radiating the cancer of the subject.

### FIELD OF THE INVENTION

The present invention relates to a method for treating cancer.

### BACKGROUND OF THE INVENTION

Lung cancer has been the most-diagnosed cancer in the world since 1985, and by 2002, there were 1.35 million new cases, representing 12.4% of all new cancers. The 5-year survival rate in the United States is 15% that is the best recorded at all population levels (Parkin D M, et al. CA Cancer J Clin 2005; 55:74-108). Non-small cell lung cancer (NSCLC) is the common type of lung cancer, even though when has been diagnosed at early stages, NSCLC has often begun to metastasize, leading to frequent systemic relapses and a poor prognosis (Hirsch F R, et al. Clin Cancer Res 2001; 7:5-22). The majority of NSCLC patients are not eligible for surgical resection, and ionizing radiation (IR) is one of the most commonly used and efficacious strategies for lung cancer therapies (Pfister D G, et al. J Clin Oncol 2004; 22:330-53). However, there are some limitations in the clinical efficacy of radiotherapy, such as normal tissue tolerance and inherent tumor radio-resistance those could hinder successful outcome. Therefore, development to achieve better effective strategy and to lower toxicity is urgent.

The molecular processes involved in the responses of neoplastic epithelial cell to radiation are unclear. Apoptosis, the cell death mediated by a cascade of caspases, plays only a partial role in the killing of neoplastic epithelial cells by radiation. Another type of programmed cell death, autophagy, has been reported to be initiated by irradiation (Paglin S, et al. Cancer Res 2001; 61: 439-444). Autophagy is characterized by sequestration of bulk cytoplasm and organelles in autophagic vesicles (also named as autophagosomes) that are later fused with lysosome to generate autolysosome and are degradated by the cells own lysosomal system. Autophagy is a multi-step process, and various signaling pathways have been implicated in its up- or down-regulation. Bcl-2 has also been shown to regulate autophagy in cancer cells, could activate the kinase mTOR leading to suppression of autophagy (Meijer A J, et al. Int J Biochem Cell Biol 2004; 36:2445-62). The other autophagy-regulating protein is beclin-1, a product of a tumor suppressor gene, which is involved in the elimination of cancer cells by triggering non-apoptotic cell death (Pattingre S, et al. Cancer Res 2006; 66:2885-88). Recently, many reports have demonstrated that irradiation and chemotherapy induced autophagic cell death in some cancer cell types (Paglin S, et al. Cancer Res 2001; 61: 439-444.& Kanzawa T, et al. Cancer Res 2003; 63:2103-08).

Berberine is an isoquinoline derivative alkaloid isolated from many medicinal herbs, such as Rhizoma coptidis. It is widely used in traditional Chinese medicine for the treatment of inflammation diseases (Ivanovska N, et al. Int J Immunopharmacol 1996; 18:553-61). In recent years, berberine has been reported to have a wide range of pharmacological effects, including interaction with DNA to form complexes, arresting effect of cell cycle progress, inhibition of tumor cells proliferation (Anis K V, et al. J Pharm Pharmacol 2001; 53:763-68.& Jantova S, et al. J Pharm Pharmacol 2003; 55:1143-49). Recently, it had also been reported the inhibitory effect of berberine on the invasion of human lung cancer cell. (Peng P L, et al. Toxicol Appl Pharmacol 2006; 214:8-15).

### SUMMARY OF THE INVENTION

The present invention provides a method for treating a subject suffering cancer, comprising administering an effective amount of berberine or its acid or ester derivates to the subject in need of such treatment, and radiating the cancer of the subject.

### BRIEF DESCRIPTION OF THE DRAWINGS

FIG. 1 shows the effect of barbering combined with radiation on the viability of A549. MTT assay for cell viability analysis was performed in (A) A549 cells treated with DMSO and the indicated concentrations of berberine combined with ionizing radiation for 24 h, and in (B) cells treated with DMSO, and the indicated concentrations of berbering alone or combined with IR (6 Gy) for 24 h and 48 h. (C) Cells were treated with radiation alone or in combination with berberine and then subjected to a fluorescent microscope.

FIG. 2 shows effect of barbering, ionizing radiation (IR) or both on cell-cycle distribution of A549 cells. Cells were treated with IR (6 Gy) alone or in combination with berbering for 24 h and then stained with propidium iodide for FACScan analysis.

FIG. 3 shows examination of biochemical apoptotic hall marks in A549 cells treated with berbering, ionizing radiation (IR), or both. (A) A549 cells were stained with DAPI and observed by fluorescent microscope 24 h. (B) DNA was extracted from the cells with the treatments as described in (A), and subject to electrophoresis. (C) Cell lysates were prepared from the cells with the treatments as described in (A), and analyzed by Western blotting for the activation of caspasa-3 and PARP.

FIG. 4 illustrates development of autophagy in A549 cells treated with radiation and berberine. (A) Electron micrographs showed the ultrastructure of A549 cells exposed to radiation alone or combined with berberine. (B) Acridine orange staining showed acidic vascular organelles formation in a dose-dependent manner when cells were treated with berberine alone or combine with IR (6 Gy) for 24 h. Bar, 25 μM. (C) The proportion of acridine orange-positive cells was quantitated by fluorescence-activated cell-sorting analysis. (D) Cell lysates were prepared from the cells treated with radiation alone, berberine alone, or IR combined with berberine for 24 h, and then subjected to immunoblotting for LC3-I, LC3-II. The lower panel shows the results of densitometric quantification.

FIG. 5 shows effects of co treatment of berberine and irradiation on mitochondrial transmembrane permeability transition. (A) A549 cells were untreated (control), treated with ionizing radiation (IR) (6 Gy), or IR combined with berberine for 24 h, and then A m was assessed with JC-1 fluorescence by flow cytometry. (B) Effect of pan-caspase inhibitor (z-VAD-fmk), autophagy inhibitor (3-MA), and beclin-1 siRNA on the cell viability of A549 cells cotreated with IR and berberine. (C) Cells were treated the same as with (B), and then subjected to clonogenic survival assay for long-term survival evaluation. Survival curve of A549 cells with different treatments were assayed by linear-quadratic model curve it.

FIG. 6 illustrates combination of radiation and berberine abridged the growth of long tumor xenograft. (A) experimental design of Lewis lung carcinoma (LLC) model in mice. LLC cells were implanted to C57BL/6 mice 10 days before the ionizing radiation (IR) (8Gy) therapy that was given as one dose. Animals were divided into six groups: untreated, IR alone, berberine 1 mg/kg, berberine 2 mg/kg, IR combined with 1 mg/kg berberine, IR combined with 2 mg/kg berberine. Berberine was provided intraperitoneally twice weekly, to the indicated groups 1 day before the IR treatment and combined for 28 days. (B) Tumor growth curve. (C) Tumor appearance was shown. (D) Immunohistochemical analysis of becline-1 and bel-2 proteins in xenografts. The tumor tissues were harvested at the indicated time point after tumor cell inoculation and processed for becline-1 and bel-2 staining.

### DETAILED DESCRIPTION OF THE INVENTION

Although many prior arts have demonstrated that in some settings combination treatment was often more effective than radiation therapy alone (Ryu M R, et al. Mol Cells 2005; 19:143-48.& Raben D, et al. Clin Cancer Res 2005; 11:795-805), there is no research yet reporting the effect of combining berberine with irradiation. In the present invention, the impact of berberine on radio-sensitivity was investigated by looking at several relevant evidences, including cell viability, cell cycle progression and clonogenic survival assay in A549, a human non-small cell lung cancer cells with a radio-resistant capacity. The enhancing effect of berberine on radiotherapy was also tested in an animal model. The present invention shows that berberine sensitized tumor cells to ionizing radiation by inducing the mechanism of autophagic cell death. These results of the example support that the use of berberine could be regarded as a supplement during radiotherapy to achieve synergistic therapeutic effect.

The present invention provides a method for treating a subject suffering cancer, comprising administering an effective amount of berberine to the subject in need of such treatment, and radiating the cancer of the subject. In a preferred embodiment of this invention, the cancer is lung cancer; in particular, the lung cancer is non-small cell lung cancer. The subject set forth is human.

The method of this invention induces autophagic cell death. The effective amount of berberine is from 0.1 to 60 μM. The more effective amount of berberine is from 1 to 40 μM. The best effective amount of berberine is from 2 to 10 μM. Combined barbarine and radiation to treat lung cancer is not only enhance the lethality of radiation but also lower the dose of radiation.

### EXAMPLE

### Cell Line, Culture and Chemicals

A549, a human lung cancer cell line, MRC-5, normal human lung cell line and Lewis lung carcinoma cell line (LLC) were obtained from ATCC (Manassas, Va.) and cultured in DMEM (Life Technologies, Grand Island, N.Y.). All cell cultures were maintained at 37° C. in a humidified atmosphere of 5% CO₂. Berberine (C₂₀H₁₈CINO₄) (Sigma St Louis, Mo.). For the treatment, medium was removed and replaced with a fresh medium containing DMSO (final concentration<0.1%) or different concentrations of berberine. Z-VAD-Fmk (Alexis-Biochemical, San Diego. CA), 3-methyladenine (3-MA) (Sigma, Aldrich), beclin-1 siRNA (Santa Cruz biotechnology, USA).

### Ionizing Radiation Modalities

Cells were cultured in phenol-red-free medium and irradiated with a 100 kV industrial X-ray machine (Varian, 21-EX). The radiation was delivered as a single dose ranging from 2 to 8 Gy in an appropriate field size at a dose rate of 400 cGy/min. A 3-cm polystyrene block was used under the petri dishes during each irradiation to allow homogeneous backscattering radiation. Control cells were removed from the incubator and placed for the same period of time under the IR source but without radiation treatment. In the combined treatment mortality tests, berberine was added 2 h prior to irradiation.

### Berberine Radio-Sensitized Effect in Non-Small Cell Lung Cancer Cells

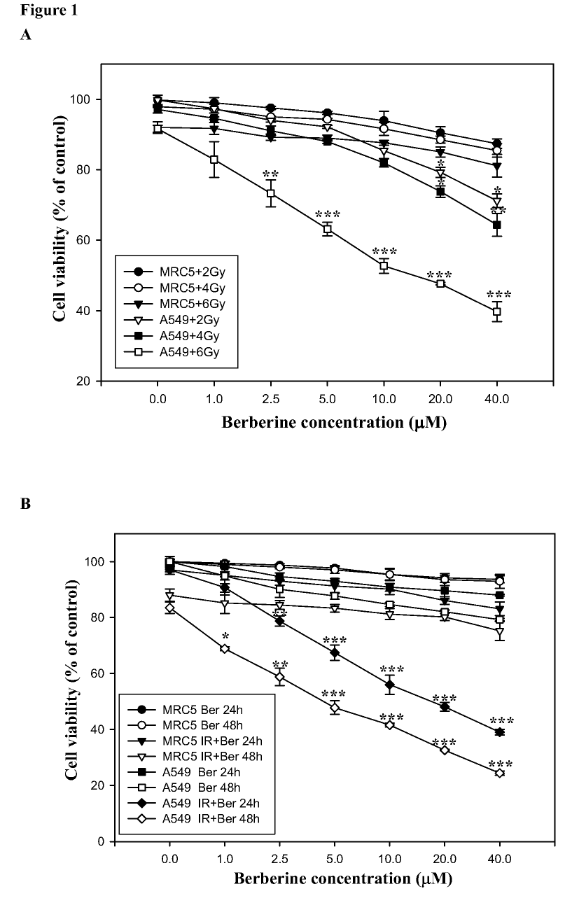

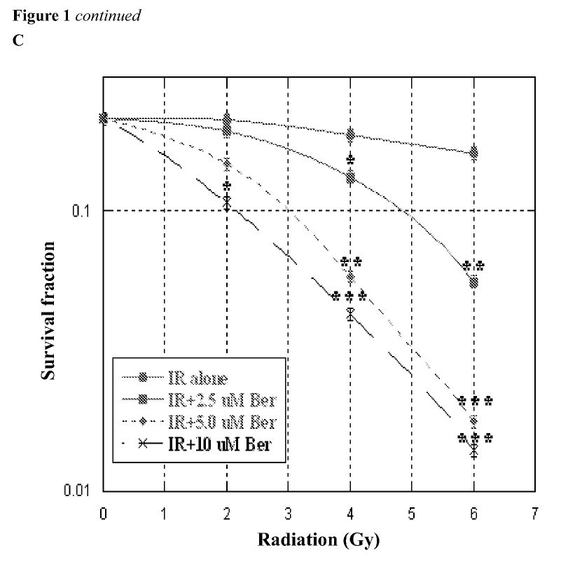

The enhancing effect of berberine on radio-sensitivity in NSCLC was performed by treating A549 with various concentrations of berberine alone or combined with different dose of IR at 37° C. for 24 h or 48 h followed by MTT assay for cell viability analysis. Thereafter, the medium was changed and incubated with 100 μl MTT (Sigma St Louis, Mo.)/well for 4 h, which was solubilized in isopropanol, and measured spectrophotometrically at 563 nm. Combination effect is conspicuous at 6 Gy IR (FIG. 1A). As shown in FIG. 1B, berberine (2.5 to 40 μM) alone remained non-cytotoxic to the cells for up to 24 h, and revealed slight cytotoxicity (20 and 40 μM) when the treatment was prolonged to 48 h. The cell viability was decreased as IR was applied to the cells incubated with berberine. This dose and time dependent synergic-killing effect was also observed in LLC cells (data not show) but not in human fetal lung fibroblasts, MRC-5. To further ascertain the effect of berberine on the lethality of IR, clonogenic survival assay was performed to examine long term survival. IR or berberine-treated cells (1000 cells) were plated onto 10 cm tissue culture dish in five replicates. After 14 days, colonies were fixed with methanol, stained with 0.25% crystal violet, and counted if they were consisted of more than 50 cells. The fraction of colonies survived from the irradiation was normalized to that of the corresponding control. The curves were fitted to the data using linear-quadratic model. The data were depicted in FIG. 1C after normalization. The D₀ value (dose of radiation producing a 37% survival rate) for the cells exposed to IR alone was 5.29 Gy, which was decreased dose-dependently as berberine was added to the cells at the same time. The D₀ values were 4.01, 2.70 and 1.24 Gy for berberine of 2.5, 5.0 and 10 μM respectively (Table 1). The addition of 10 μM berberine led to a 4.2-fold decrease in the D₀ value, indicating that berberine could be used as a supplement to enhance the lethality of radiation or to lower the dose of radiation.

TABLE 1
Radiation inactivation estimates of A549 cells treated with radiation
alone or in combination with berberine.

| Treatment | SF2 | D₀ (Gy) |
|---|---|---|
| Radiation alone | 0.4226 | 5.29 |
| IR + 2.5 μM Ber | 0.3184 | 4.01 |
| IR + 5.0 μM Ber | 0.2152 | 2.70 |
| IR + 10 μM Ber | 0.0991 | 1.24 |

### Berberine Radio-Sensitized A549 Cells by Inducing Cell Cycle G₂/M Arrest

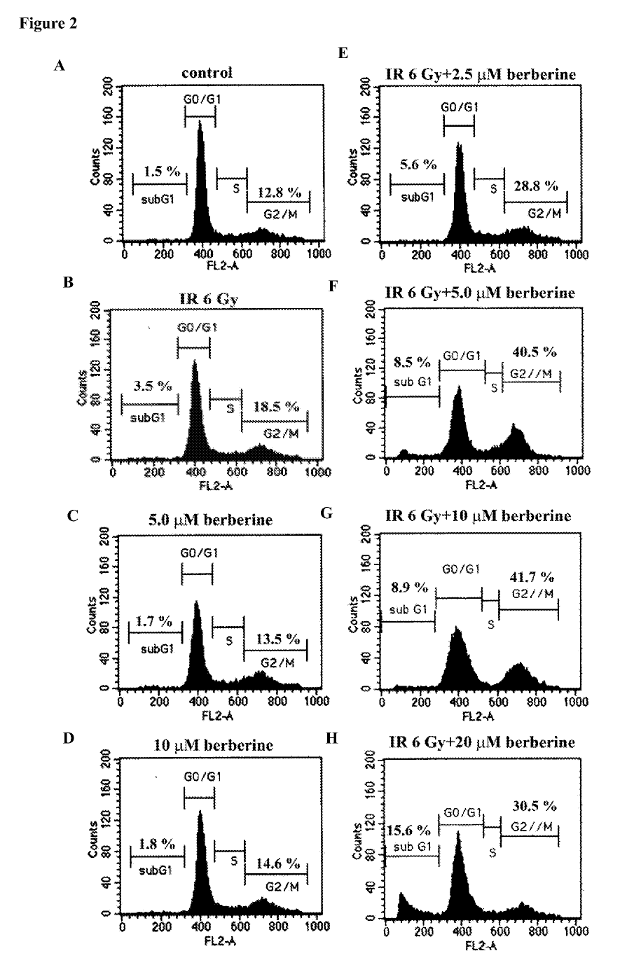

The long term survival of A549 cells was affected more severely (FIG. 1C) by the co-treatment of IR and berberine than the short term cell viability (FIGS. 1A and 1B), indicating the treatment caused a long term damage to the cells. In order to understand the underlying mechanism, the cell cycle distribution of A549 cells treated with berberine at non-cytotoxic concentration (1 to 10 μM), IR or co-combination were investigated. Flow cytometry was performed by propidium iodide staining (Roche Molecular Biochemical, Indianapolis, USA) according to the manufacturer's instructions. Untreated and treated cells were washed in PBS and fixed in ice-cold ethanol. Fixed cells were pelleted and resuspended in 500 μl of PBS. Then, the cells were stained with propidium iodide and analyzed for cell cycle distribution by flow cytometry with a FACStar caliber (Becton Dickinson) cell sorter. The percentage of each phase was evaluated by Cell-Quest software of the histograms. The results showed that the treatment of IR (6 Gy) alone did not influence the cell population of each phase. With the supplement of berberine, there was an increase in the cell number of G₂/M phase (28.8%, 40.5% and 41.7% for 2.5, 5.0 and 10 μM of berberine respectively, FIG. 2), demonstrating a G₂/M arrest that is important for IR sensitivity (Milas L, et al. Semin Radiat Oncol 1999; 9:12-26.). This dose-dependent G₂/M block was accompanied by a slight elevation in the sub-G1 population, an index of apoptotic cell death, to 8.5% when cells were treated with 5.0 μM berberine along with IR. Nevertheless, a higher concentration of berberine (10 μM) caused a decrease in G₂/M and an increase in sub-G1 proportion as compared to the data of 5.0 μM berberine treatment group under IR exposure. Thus, the effect of berberine on sensitizing A549 cells to IR was attributed to the inductions of G₂/M arrest and, partially apoptosis (Table 2).

TABLE 2
Effect of ionizing radiation or in combination with berberine on cell cycle
distribution.

| Treatment | sub G1 | G0/G1 | S | G2/M |
|---|---|---|---|---|
| untreated | 1.5 ± 0.11 | 71.2 ± 5.15 | 14.5 ± 1.89 | 12.8 ± 1.05 |
| IR alone | 3.5 ± 0.15 | 62.3 ± 5.02 | 15.7 ± 1.92 | 18.5 ± 1.75 |
| IR + 2.5 μM | 5.6 ± 0.18 | 45.5 ± 4.96 | 20.1 ± 2.26 | 28.8 ± 2.91 |
| Ber | | | | |
| IR + 5.0 μM | 8.5 ± 0.22 | 36.2 ± 4.15 | 14.8 ± 2.05 | 40.5 ± 4.55 |
| Ber | | | | |
| IR + 10 μM | 8.9 ± 0.25 | 33.9 ± 4.01 | 15.5 ± 1.81 | 41.7 ± 3.91 |
| Ber | | | | |
| IR + 20 μM | 15.6 ± 1.26 | 37.2 ± 4.27 | 16.7 ± 2.33 | 30.5 ± 3.18 |
| Ber | | | | |

### Apoptosis was not the Main Cell Death Pattern in the Cell-Killing Effect of Co-Treatment of Berberine and IR

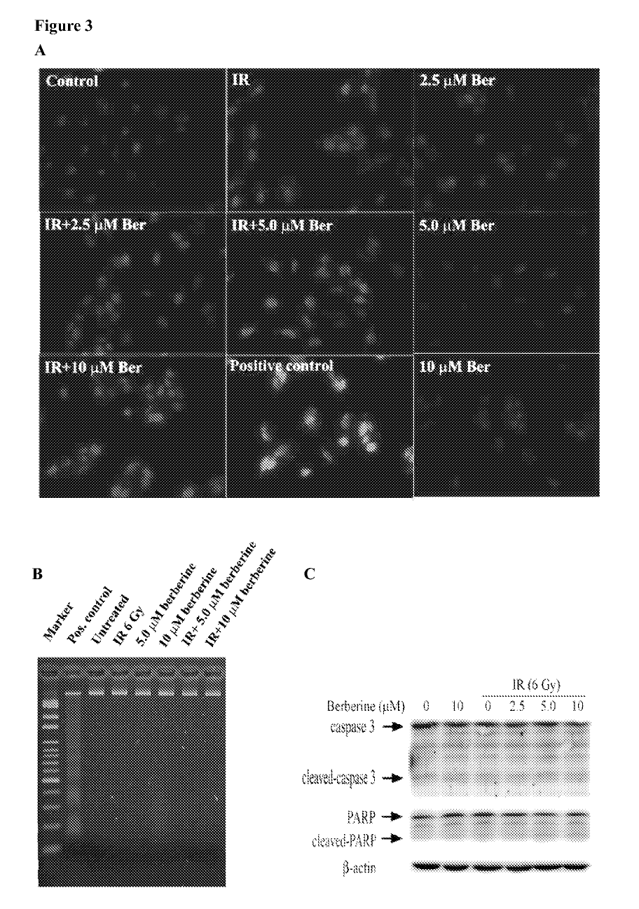

The role of apoptosis in the sensitivity of A549 cells to IR induced by berberine was further clarified by DAPI stain. DNA extraction and electrophoresis on agarose gel were carried out as described previously (Solary E, et al. Therapie 2001; 56:511-18). After the indicated treatments, cells were fixed with 3 mL of 4% paraformaldehyde. To stain the cells, 500 μL of a 0.5 μg/mL chilled solution of DAPI (Invitrogen) stain was added and allowed to sit for 5 min. The cells were then rinsed with PBS solution and counted under a fluorescent microscope. For positive control of DNA fragmentation and DAPI stain, A549 cells were treated with 30 μM etoposide for 24 h. The results revealed no significant biochemical and morphological changes that are associated with apoptosis 24 h after irradiation (6 Gy) alone, and chromatin condensation were detected only in a small fraction of cells (FIG. 3A). The number of apoptotic cells increased only slightly upon the co-treatment of berberine (>5.0 μM) with IR. This contention was further confirmed by the lack of apoptotic DNA fragmentation and activation of caspase-3 and poly-(ADP-ribose) polymerases (PARP) as analyzed by DNA electrophoresis and immunoblotting (FIG. 3B, 3C). After the indicated treatment, the resultant supernatants were separated in 12%˜18% polyacrylamide gels. The blot was incubated with a polyclonal antibody against LC3 (gift from T. Yoshimori), PARP, caspase-3, bcl-2, beclin-1 and β-actin (Santa Cruz biotechnology, USA) for 2 h, and then with an appropriate peroxidase-conjugated secondary antibody (Sigma) for 1 h at 37° C. Signal was developed by 4-chloro-1-napthol/3,3-o-diaminobenzidine, and the relative photographic density was quantitated by scanning the photographic negatives with a gel documentation and analysis system (Alpha-Imager 2000, San Leandro, Calif., USA). These results indicated that apoptosis was not the main mechanism in radio-sensitizing effect of berberine, since the proportion of apoptotic cell death did not reflect the additive cytotoxicity of berberine and IR (FIG. 1).

### Co-Treatment of Berberine and IR Induced Autophagic Cell Death in A549 Cells

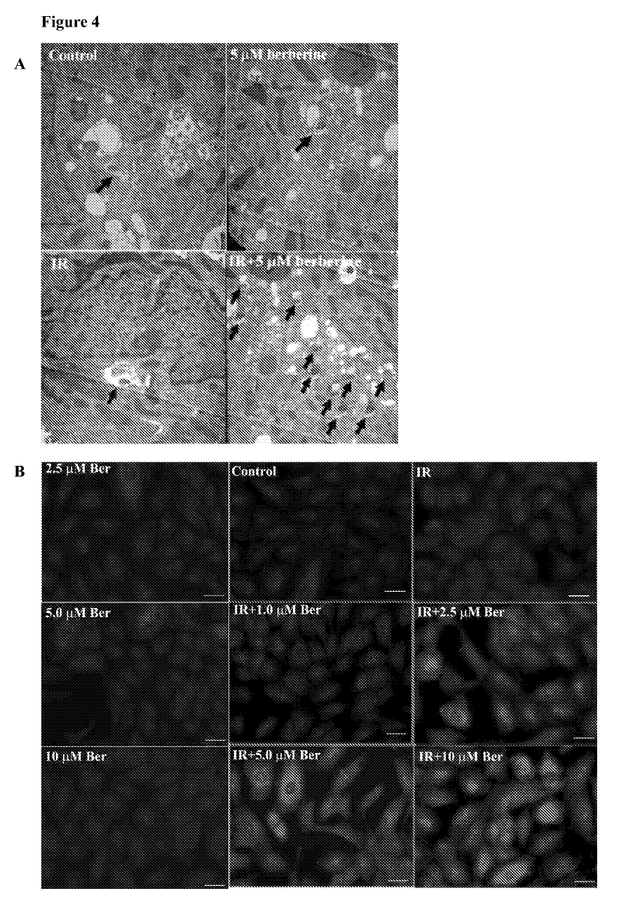

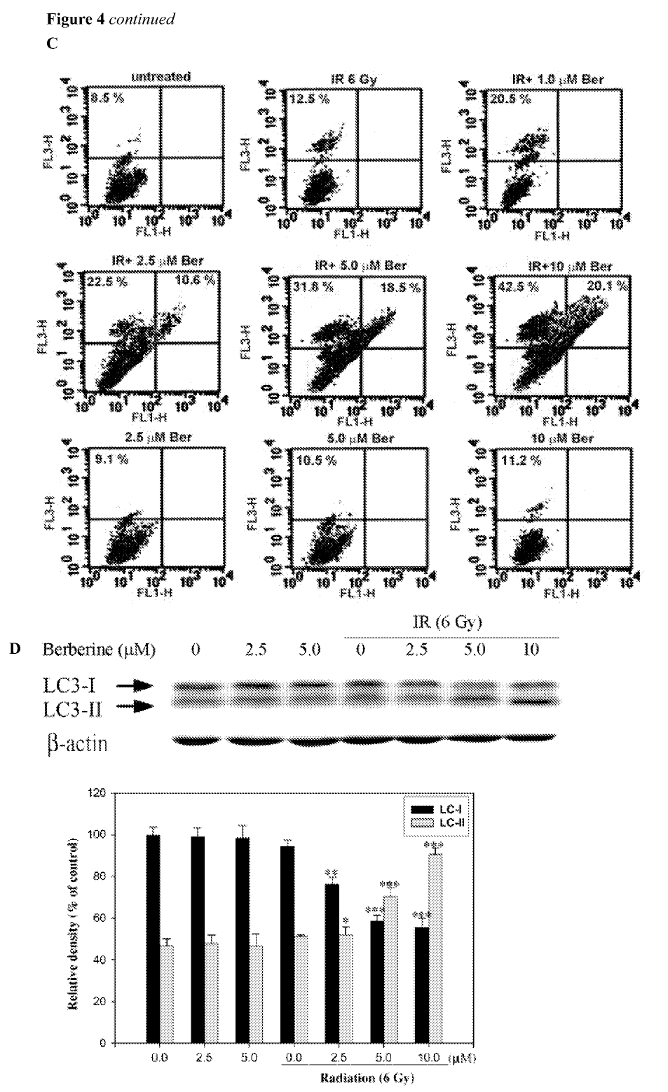

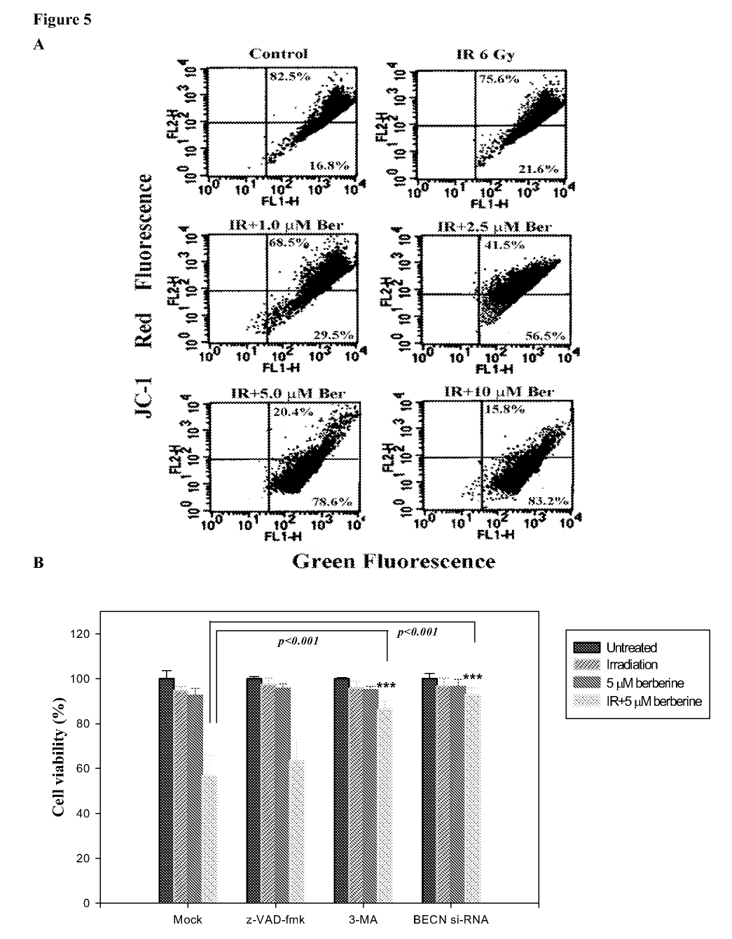

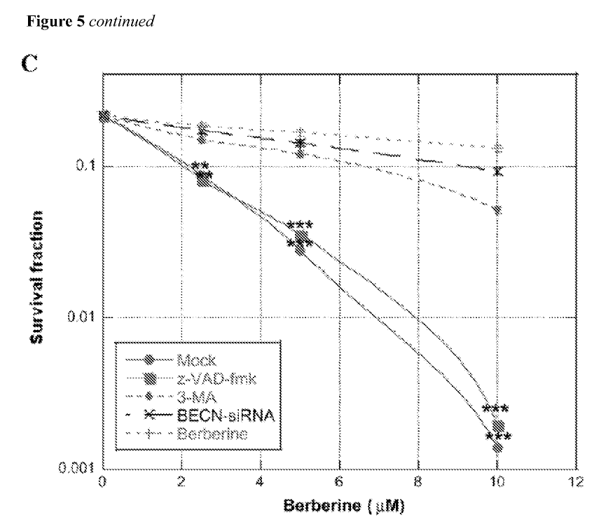

Autophagic cell death has been recognized as programmed cell death type II, it has been detected in a variety of cancer cells treated with radiation or chemotherapy. Therefore, it had been examined whether IR treatment combined with berberine induced autophagic cell death in A549 cells by electron microscopic analysis (FIG. 4A). Cells were harvested by trypsinization, and fixed with 2% glutaraldehyde, 4% paraformaldehyde and 1% tannic acid in 0.1 mol/L cacodylate buffer, pH 7.4, for 25 h. The cells were stained with an osmium-thiocarbohydrazide-osmium (OTO). After staining, the cells were dehydrated in a graded series of 70% to 100% EtOH and then immersed serially with 1:1 hexamethyldisilazane and absolute ethanol. One micrometer thin sections were cut, and the gels were coated with 500 Å of gold in a JEOL Vacuum sputter coater and viewed in a JEOL T300 electron microscope with scanning attachment (JEOL, Tokyo, Japan). Numerous empty vacuoles and autophagic vacuoles contained intact cytoplasmic structure, lamellar structure, or residual digested material (arrows) were observed in A549 cells co-treated with IR and 5.0 μM berberine for 24 h. Autophagy is characterized by the development of AVOs, which is measured by vital staining of acridine orange. Acridine orange (Polysciences, Warrington, Pa.) was added to cells at a final concentration of 1 μg/mL for a period of 15 min. Pictures were taken with a fluorescent microscope (Nikon TE-300) equipped with a 490-nm band-pass blue excitation filters, a 500-nm dichroic mirror, and a 515-nm-long pass-barrier filter. To quantitate acidic vesicular organelles, Stained cells were collected for the FACScan (Becton Dickinson, San Jose, Calif.) using CellQuest software (Becton Dickinson). Acridine orange-positive cells with bright red fluorescence were detected among A549 cells 24 h after co-treated with berberine and IR (6 Gy), whereas they were sparse among non-treated cells or irradiated cells (FIG. 4B). As analyzed by fluorescence-activated cell sorting analysis, the proportion of acridine orange-positive cells increased from 12.5% to 50.3% and 62.6% in A549 cells after 6 Gy irradiation combined with berberine of 5.0 and 10 μM respectively, whereas there was no apparent increase among irradiated cells (from 8.5% to 12.5%; FIG. 4C).

LC3 is localized in autophagosome membranes during amino acid starvation-induced autophagy (Mizushima N, et al. J Cell Biol 2001; 152:657-68.). Recent investigation showed that there are two forms of LC3 proteins in cells. LC3-I, the cytoplasmic form of LC3, is processed into LC3-II that is associated with the autophagosome membrane. Therefore, the amount of LC3-II is correlated with the extent of autophagosome formation. It had been examined the expressions of LC3-I (18 kDa) and LC3-II (16 kDa) in non-small lung cancer cells treated with berberine and IR. As shown in FIG. 4D, the expression of LC3-II increased dose-dependently in A549 cells 24 h after exposed to the combined treatment with increasing concentrations of berberine.

### Co-Treatment of Berberine and Irradiation Caused Mitochondrial Disruption

Berberine has been reported to induce cell death in several cancer cells via mitochondrial disruption (Lin J P, et al. World J Gastroenterol 2006; 12:21-28.). Therefore, mitochondrial membrane potential was next measured with JC-1. Mitochondria membrane potential (Δ Ψₘ), the lipophilic cationic probe fluorochrome JC-1 (Invitrogen) was used. JC-1 exhibited a potential-dependent accumulation in mitochondria indicated by a fluorescent emission shift from 530 to 590-nm. After a treatment of radiation (6 Gy) for 24 h in the absence or presence of 1.0-10 μM berberine, cells were rinsed with DMEM, and JC-1 (5 μmol/L) was loaded. Determination of Δ Ψₘ was also carried out with a FACScan Flow Cytometer. The treatment of 1.0, 2.5, 5.0 and 10 μM berberine combined with irradiation (6 Gy) to A549 cells for 24 h induced a great loss of membrane potential that was not significantly affected by irradiation alone (FIG. 5A). It has been reported that depolarized mitochondria would move into autophagic vacuoles in response to appropriate stimulation; thus, mitochondria dysfunction may be a point of overlap between apoptotic and autophagocytic processes (Bras M, et al. Biochemistry (Mosc) 2005; 70:231-39). To further demonstrate the type of berberine radio-sensitized cell death, caspase and autophagy inhibitors were used in inhibition assay. As shown in FIG. 5B, z-VAD-fmk (caspase inhibitor) did not significantly affect the cell viability of A549 cells co-treated with berberine and IR (p=0.15). On the other hand, beclin-1 siRNA and 3-MA (autophagy inhibitor) blocked the cell death induced by 5.0 μM berberine combined with 6 Gy IR (p<0.001). As a consequence, berberine sensitized A549 cells to IR damage via inducing autophagic cell death.

### Combination of Radiation and Berberine Abridged the Growth of Lung Tumor Xenograft

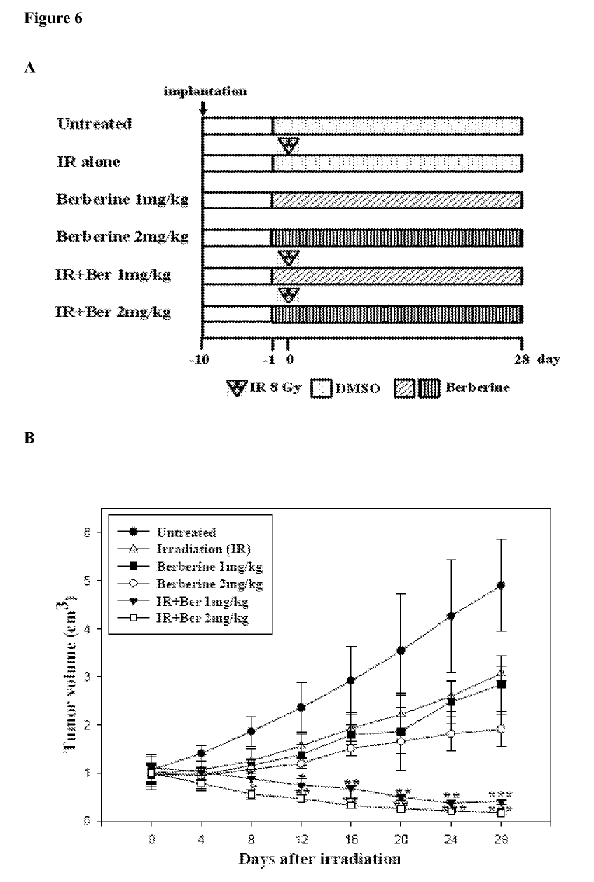

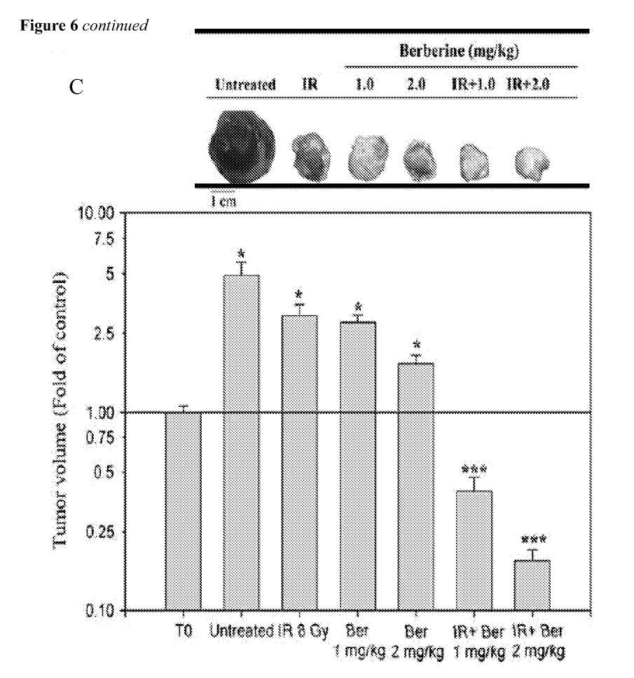

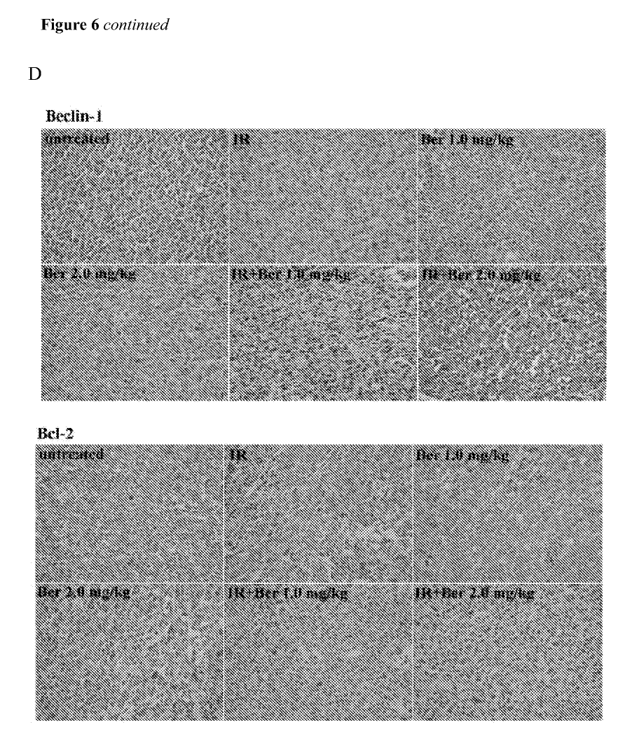

The application of the additive cytotoxicity of IR combined with berberine in cancer treatment was further evidenced in an in vivo Lewis lung carcinoma (LLC) model in mice (Camphausen K, et al. Cancer Res 2001; 61:2207-11). Male C57BL/6 mice aged 4-6 weeks (National Taiwan University Animal Center, Taiwan) were used. To form a tumor xenograft, Lewis lung cells (2×10⁶) were injected s.c. into the right hind limb. Animals were randomized into six groups: control, irradiation (IR) alone, berberine (1.0, 2.0 mg/kg) alone, and IR combined with berberine (1.0, 2.0 mg/kg). For tumor irradiation, mice were immobilized in a customized harness that exposed the right hind leg while shielding the remainder of the body by 3.5 cm of lead. Mice were exposed to a single dose of 8 Gy. Berberine was supplemented (1.0, 2.0 mg/kg, i.p.) to animals 1 day before the radiation exposure, and then twice a week for 4 weeks. To obtain tumor growth curves, three orthogonal tumor diameters were measured every 4-day intervals with a vernier caliper, and the mean values were calculated. The animals were killed (n=5 per group) at the indicated time points, and the primary tumor was dissected and examined. Primary tumor growth delay was measured by calipers in two dimensions, and the volumes were calculated according to the formula: tumor volume=(width) 2×length×0.52. LLC cells were injected s.c. into the right thighs of C57BL/6 mice 10 days before the IR therapy. The animals were randomized into six treatment groups: untreated, 8 Gy radiation in one dose, berberine alone (1.0, 2.0 mg/kg i.p., twice weekly), and berberine combined with IR treatment (FIG. 6A). Berberine was supplemented to the indicated groups one day before the IR treatment and continued for 4 weeks. The tumor volume at the primary site of each group was measured before treatment, and the average tumor volume was designated as T₀. When the animals were sacrificed at the end of experiment, the xenografts were removed, and the tumor volume was determined and presented as fold of T₀. The blood samples were collected for biochemical analysis. The serum levels of AST, ALT, urea nitrogen, creatinine, complete blood cell counts, triglyceride and total cholesterol showed no difference among groups. As shown in FIG. 6, the tumor size of the untreated animals at the end of the experiment was 5 fold of that before the treatment. The growth of the xenograft was slowed down when the animals were treated with IR or berberine alone, whereas the combination treatment groups displayed a considerable reduced size as shown in the tumor growth curves (FIG. 6B). Berberine at 1.0 and 2.0 mg/kg, as used with IR, has shrunk the tumor volume down to approximately 48% and 22% of that before the treatment (FIG. 6C, p<0.001). Furthermore, immunohistochemistry was performed to detect the level of beclin-1 and bcl-2 proteins in the xenograft. Primary tumors were fixed and then embedded in paraffin. The sections were stained with anti-bcl-2 mouse-monoclonal antibody (1:100, Santa Cruz Biotechnology, Santa Cruz, Calif.) or anti-beclin-1 rabbit polyclonal antiserum (1:50, Santa Cruz Biotechnology, Santa Cruz, Calif.). The sections were then incubated with blocking buffer for 40 min at RT followed by incubation with the primary antibody over night at 4° C. Following incubation with the secondary antibody, the ImmunoCruz system (Santa Cruz Biotechnology, Santa Cruz, Calif.) was used. Sections were counterstained with hematoxylin. As depicted in FIG. 6D, the results showed a remarkable increased in beclin-1 protein expression in the co-treatment groups. Besides, bcl-2 protein expression was reduced in berberine alone and co-treatment groups. Given the above, these figures demonstrated that autophagy was involved in the co-treatment-induced tumor elimination. Consequently, berberine was able to reinforce the killing effect of irradiation in local tumor growth.

### Statistical Analysis

Statistical significances were analyzed by student t-test or one-way analysis of variance (ANOVA) with post-hock Dunnett's test. P-value≦0.05 was considered statistically significant (Sigma-Stat 2.0, San Rafael, Calif., USA). Survival fraction curve was analyzed by KaleigaGraph 4.0 then fitted to the data using linear-quadratic model. Combination index was analyzed by CalcuSyn.

1. A method for treating a subject suffering cancer, comprising:
(a) administering an effective amount of berberine or its acid or ester derivates to the subject in need of such treatment, and
(b) radiating the cancer of the subject.

2. The method of claim 1, wherein the cancer is breast cancer, lung cancer, liver cancer, rectal cancer, head or neck cancer.

3. The method of claim 2, wherein the cancer is lung cancer.

4. The method of claim 3, wherein the lung cancer is non-small cell lung cancer.

5. The method of claim 1, wherein the subject is human.

6. The method of claim 1, which induces autophagic cell death.

7. The method of claim 1, where the effective amount of berberine is from 0.1 to 60 μM.

8. The method of claim 7, where the effective amount of berberine is from 1 to 40 μM.

9. The method of claim 8, where the effective amount of berberine is from 2 to 10 μM.

10. The method of claim 1, which enhance the lethality of radiation.

11. The method of claim 1, which lower the dose of radiation.

12. The method of claim 1, wherein the berberine or its acid or ester derivates are selected from the group consisting of Berberidaceae, Ranunculaceae, Papaveraceae, Menispermaceae, and Rutaceae.
